(19) United States
(12) Patent Application Publication
(10) Pub. No.: US 2009/0182288 A1
(43) Pub. Date: Jul. 16, 2009

(54) Flexible Medical Cannula

(76) Inventor: David B. Spenciner, North Attleboro, MA (US)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 12/348,736

(22) Filed: Jan. 5, 2009

Related U.S. Application Data
(60) Provisional application No. 61/019,054, filed on Jan. 4, 2008.

(51) Int. Cl. A61M 5/00

(52) U.S. Cl. 604/264

(57) ABSTRACT

The present disclosure relates to a cannula which may be flexible to completely collapse in the tissue and still allow instruments to pass through the portal. The cannula may include a distal ring, which may aid to prevent inadvertent removal. In addition, the cannula may include a proximal spring for providing tension between the distal and proximal portions of the cannula.

### CROSS-REFERENCE TO RELATED APPLICATIONS

The present application claims the benefit of the filing date of U.S. Provisional Application Ser. No. 61/019,054, filed on Jan. 4, 2008, the teachings of which are incorporated herein by reference.

### FIELD

The present disclosure relates to a flexible cannula for use in medical procedures.

### BACKGROUND

In arthroscopic surgery, and other medical procedures or medical fields, including endoscopy, cardiology, opthamology, plastic surgery/liposuction, etc., access to the internal structures of the body may be performed via the use of small incisions. Elongated surgical instruments may reach through the incisions, such that structures within the body may be manipulated by the surgeon to examine a site of interest, to reduce pain or perform a repair. To aid in providing access to the procedure site, insufflation of the tissue may be provided with either sterile saline (arthroscopy) or compressed gas (endoscopy), which may expand the area proximate to the site. In both of these cases, a mechanical stopper or cannula may be used to prevent pressure equalization between the inside and outside of the body.

The cannula may have relatively rigid side walls and external threads or some other mechanism to prevent inadvertent removal. Some flexible cannulae may allow instruments of various sizes to pass through. Other cannulae may also expand or shorten. However, some cannulae may have problems, including decreased ability to resist inadvertent removal and poor sealing.

### SUMMARY

The present disclosure relates to a cannula which may be flexible to completely collapse in the tissue and still allow instruments to pass through the portal. The cannula may include a distal ring, which may aid to prevent inadvertent removal. In addition, the cannula may include a proximal spring for providing tension between the distal and proximal portions of the cannula.

### BRIEF DESCRIPTION OF THE DRAWINGS

The features and advantages disclosed herein, and the manner of attaining them, may become more apparent and the disclosure may be better understood by reference to the following description of embodiments taken in conjunction with the accompanying drawings, wherein:

FIG. 1 is an example of a cannula contemplated herein.

FIG. 2 is another example of a cannula contemplated herein.

FIG. 3 is a further example of a cannula contemplated herein.

FIG. 4 is a side view of another example of a cannula contemplated herein.

FIG. 5 is an end view of the cannula illustrated in FIG. 4.

FIG. 6 is a side view of another example of a cannula contemplated herein.

FIG. 7 is an end view of the cannula illustrated in FIG. 6.

FIG. 8 is an example of a cannula contemplated herein.

FIG. 9 is another example of a cannula contemplated herein.

FIG. 10 is a further example of a cannula contemplated herein.

FIG. 11 is yet another example of a cannula contemplated herein.

FIG. 12 is a further example of a cannula contemplated herein.

### DETAILED DESCRIPTION

The present disclosure relates to a flexible cannula for use in medical procedures, including open surgery, arthroscopy, endoscopy, etc. A cannula as understood herein may be reference to a long tube. Cannulae may be inserted into various bodily cavities, ducts or vessels and may provide access to or drain fluids from these areas.

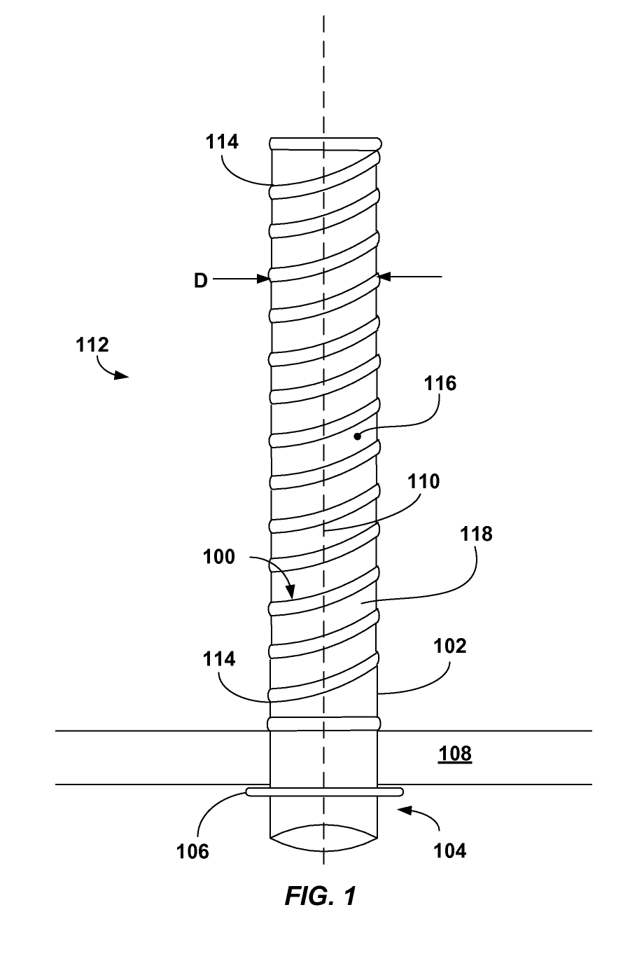

An example of the cannula mechanism contemplated herein is illustrated in FIG. 1. The cannula 100 may include an elongate, hollow, flexible sleeve 102, which may be open at both ends. Elongate may be understood herein as having a length to diameter (internal or external diameter) ratio of 2 or more to 1, i.e., the length of the cannula may be 2 times or more the diameter of the cannula. For example, the cannula may be 10 to 10,000 in length times the diameter of the cannula, including all values and increments therein. Hollow may be understood as defining a cavity within at least a portion of the cannula, such that in at least a portion of the cannula, the cannula may be tubular.

The cannula may be flexible enough such that the residual compression in soft tissue may be sufficient to clamp down on the cannula and restrict flow of, for example, gas or fluids, through the cannula. Flexibility may be indicated by the hardness, modulus of elasticity or flexural modulus of the material, as well as the stiffness of the cannula itself. In addition, it may be appreciated that the thickness of the cannula wall may be adjusted, such that for various materials, the flexibility may be adjusted. For example, the cannula may be formed from a material having a hardness of 10 to 100 Shore A durometer, including all values and increments therein, as measured by the latest revision of ASTM D2240, such as ASTM D2240-05. The cannula material may also, and/or independently, exhibit a modulus of elasticity of less than 0.1 GPa, including all values and increments in the range of 0.0001 GPa to 0.1 GPa, including all values and increments therein. The modulus of elasticity may be understood herein as the ratio of stress to strain. Furthermore, the cannula material may also, and/or independently, exhibit a flexural modulus of 1 GPa or less, including all values and increments in the range of 0.001 GPa to 1 GPa. The flexural modulus may be understood as the ratio, within the elastic limit, of flexural stress to the corresponding strain. In addition, the thickness of the cannula side wall may also, and/or independently, be in the range of 0.01 mm to 2.0 mm, including all values and increments therein, such as 0.1 to 1 mm in thickness, etc., which may depend on the materials utilized.

Cannula materials contemplated herein include thermoplastics (including thermoplastic elastomers) or thermosets. Materials may include, for example, silicone, polyvinyl chloride, latex rubber, nitrile, polyurethane, fluorpolymers such as, polytetrafluoroethylene, polyfluoroalkoxy, fluorinated ethylene propylene, nylons such as nylon 6/6, polyether block amides, polychloroprene, etc. In addition, the cannula may be formed from more than one material. For example, material blends may be utilized as well as various material layers or multiple segments each including one or more different materials.

Referring back to FIG. 1, at a distal portion 104, a shape-memory material 106 may be attached. The shape memory material may be a wire, tube, band or other geometry. The material may have a first shape, which may for example, conform to the geometry of the cannula. The shape memory material may include metal alloys such as copper-zinc-aluminum-nickel, copper-zinc-aluminum or nickel-titanium alloys or polymers. Accordingly, shape memory material may be understood herein as a material that may deform from a first shape to a second shape and substantially return to its first shape. For example, upon returning to its first shape, the material may exhibit a dimensional variation of less than +/−5% in a given dimension (e.g., width, diameter, etc.) from an initial corresponding dimension. The change in shape maybe facilitated by the application of pressure and/or temperature (either relatively hot temperatures or relatively cold temperatures). In addition, the shape memory material may be an elastomer or rubber, such as silicone, latex rubber, nitrile, polyisoprene, polybutadiene; or a fluoropolymers, such as polytetrafluoroethylene, fluorinated ethylene propylene, perfluoroalkoxy; etc., wherein the shape memory material may be, for example, a rubber o-ring.

Prior to introduction of the cannula into the skin 108, the shape memory material may be flattened or reduced in size for introduction through the skin incision. Once inside the body, the shape memory material may be allowed to expand and return to its initial shape, although the middle part of the sleeve, between the proximal and distal ends, may be flexible enough to collapse into a nearly water-tight seal due to intramuscular or other tissue pressure. The distal ring may also prevent expansion of the soft tissue (e.g. adipose tissue) which may obstruct the view during surgery and necessitates additional soft tissue removal.

The shape memory material may be larger or smaller in diameter than the diameter of the cannula itself. For example, the shape memory material may be 0.1 to 10 times the size of the cannula diameter D₁, wherein the diameter of the shape memory material D₂ may be equal to x*D₁, where x may be in the range of 0.1 to 10. It may be appreciated that in many embodiments D₂ does not equal D₁, i.e., x does not equal 1. Furthermore, where it may be preferable that the diameter of the shape memory material is greater than the diameter of the cannula, x may be greater than 1, such as in the range of 1.1 to 10, including all values and increments therein. Where it may be preferable that the diameter of the shape memory material is less than the diameter of the cannula, x may be less than 1, such as in the range of 0.1 to 0.9, including all values and increments therein. In further examples, where non-circular cannula geometries may be contemplated, it may be appreciated that rather than a diameter, a given dimension may be contemplated, such a width.

The long axis 110 of the sleeve may be nominally perpendicular to the plane of the tissue 108 and may transect it between the skin proximally and the joint space distally. The sleeve may be extra long, wherein the surgeon may trim it to an appropriate length during the operation. When the proximal part 112 of the sleeve is tugged, the distal ring 106 may pull back against the skin wall 108. This may confer the advantage of holding back soft tissue (e.g. adipose tissue) as it expands. Thus, an operation may be performed quicker and with less bulk removal because surgeon visualization may be improved. In one embodiment, there may be a proximal structure 114 which may maintain this slight pull on the sleeve. The proximal structure 114 may be, for example, a round spring that at least partially or completely encloses the sleeve and holds the proximal end open all while maintaining the tension on the sleeve. In another example, the proximal structure may be a rigid tube, having a higher rigidity than that of the sleeve. The proximal structure may be affixed to the sleeve or integrated into the sleeve.

The cannula may include a medicament such as a pain killer, antibiotic, or vasodilator, which may elute from the cannula. The medicament may be coated onto the surface 116 of the cannula or may be present in the wall 118 of the cannula. Where the medicament may be present in the wall of the cannula, the medicament may be dispersed into the material used to form the cannula or present between a first layer inner layer and a second outerlayer, wherein the medicament may pass through the outer layer or the outer layer may dissolve and the medicament may be exposed.

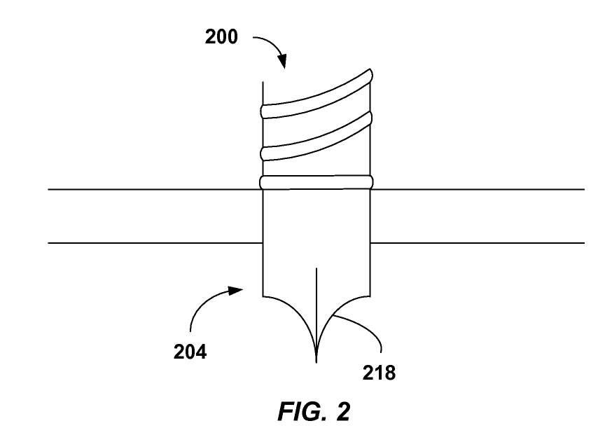

FIG. 2 illustrates another embodiment of a cannula 200 contemplated herein. The cannula may have flexible flaps 218 formed or integrated into the distal end of the cannula 204. The flaps may keep the cannula open, but decrease any outflow of fluids. As illustrated, the flaps 218, may be duck-billed in shape, however, other geometrical arrangements are considered as well. Furthermore, the flaps may be formed from a polymeric material such as silicone or other elastomeric materials, which may be flexible enough to deform but rigid enough to return to or retain their initial shape. The flaps may be provided in a neutral or closed position (as illustrated) and the application of pressure or a mechanical force from the interior of the cannula may allow for the flaps to open. However, pressure enacting upon the outer surface of the flaps may cause the flaps to close or remain closed. The flaps may also be present in combination with the shape memory material.

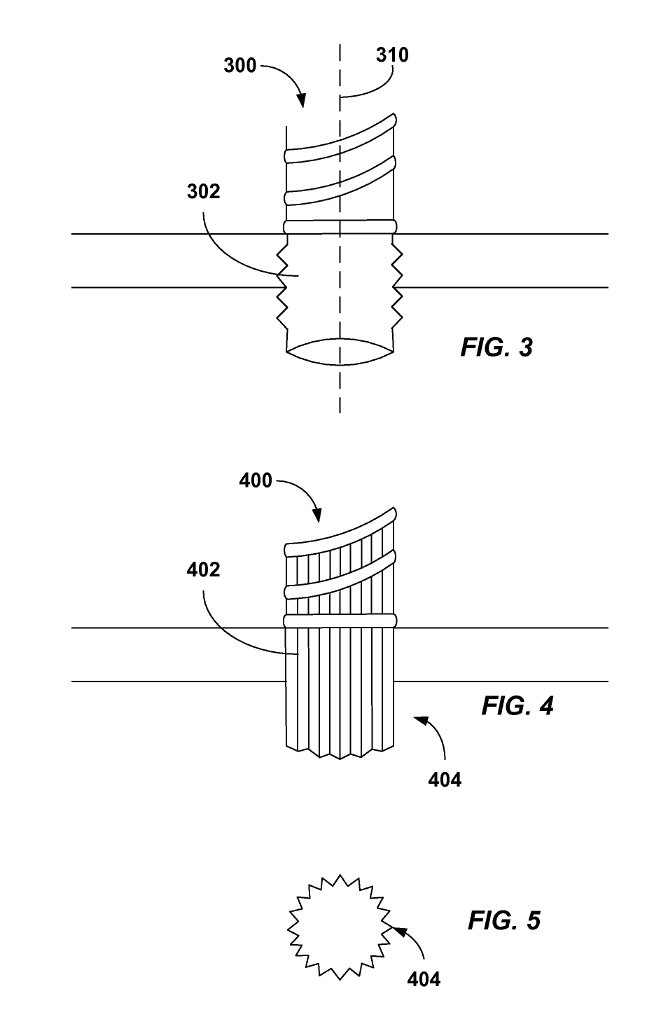

The cannula may also be extendable or expandable. FIG. 3 illustrates another embodiment of a cannula 300 contemplated herein. The cannula may include an extendable sleeve 302 to accommodate a need for extra length, such as when swelling may occur. As illustrated, the sleeve may include a corrugated portion or may be stretchable in the direction of its long axis 310. FIG. 4 illustrates another embodiment of a cannula 400, wherein at least a portion of the sleeve 402 may be corrugated or stretchable circumferentially so that the cannula would accommodate the need for an increase in the portal size. FIG. 5 illustrates an example view of the distal end 404 of the corrugated sleeve illustrated in FIG. 4.

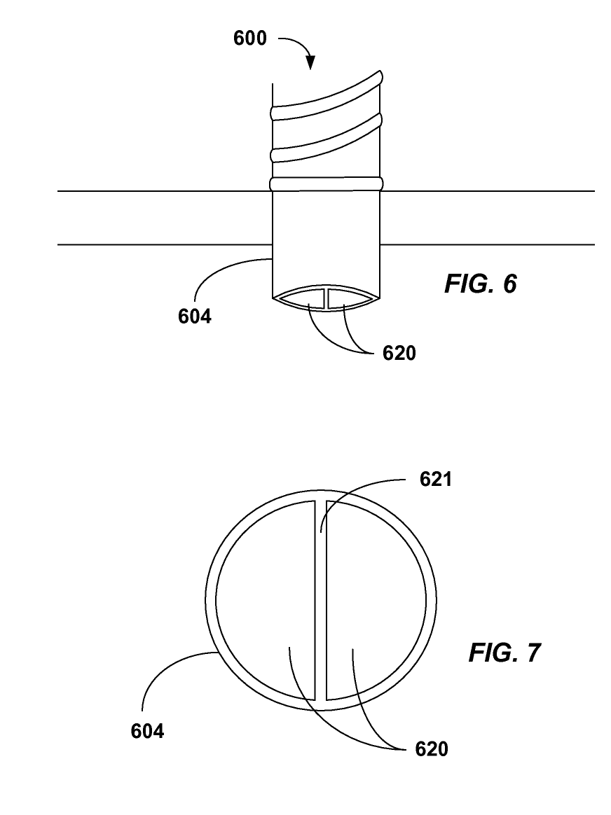

In addition, the cannula may include more than one channel provided within at least a portion of the cannula. FIG. 6 illustrates an example of such a cannula 600 including two channels 620 defined therein. A divider 621 may be provided between the channels, which may be integrated into the cannula. FIG. 7 illustrates an end view of the distal portion 604 of the cannula 600 illustrated in FIG. 6. Although, only two channels are illustrated, it may be appreciated that the sleeve may include multiple channels with dividers between them.

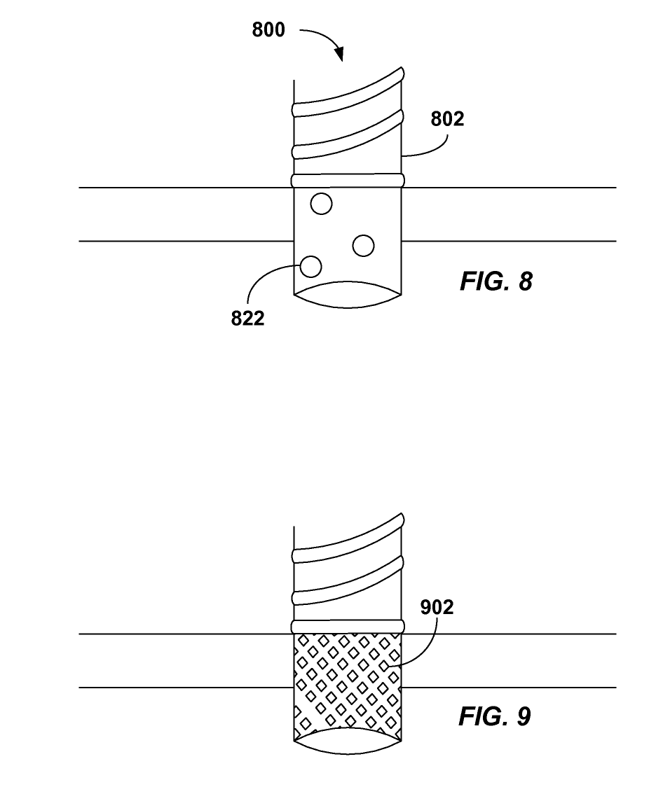

It is further contemplated herein, the cannula sleeve may be formed from a material including one or more apertures therein. For example, as illustrated in FIG. 8, a cannula 800 may be provided which may include a plurality of holes 822 in a portion of the sleeve 802 material. The holes may extend through the thickness of the cannula wall exposing the interior of the cannula to the environment. In another example, as illustrated in FIG. 9, a portion of the cannula sleeve 902 may be formed of a mesh material.

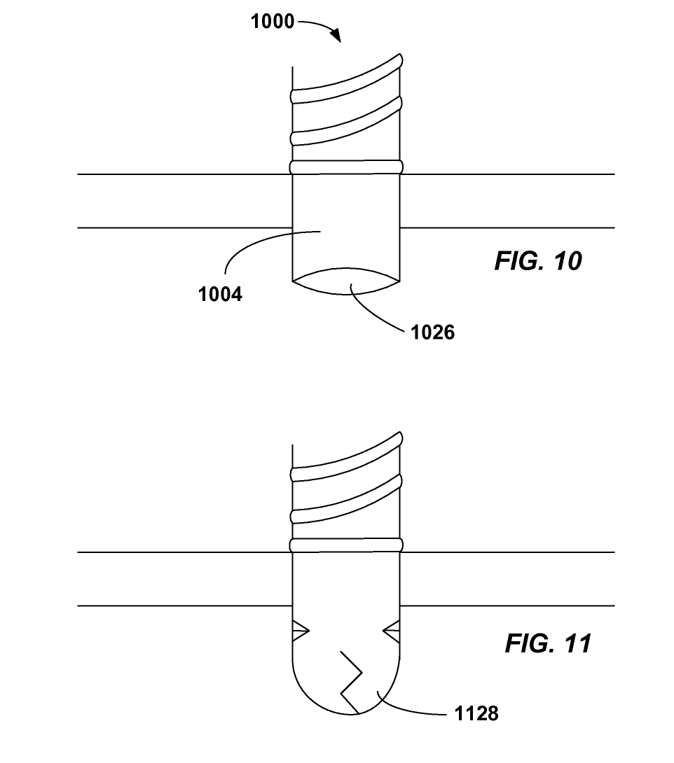

As illustrated in FIG. 10, another embodiment of the cannula 1000 may include a membrane 1026 at the distal end 1004 that may dissolve in a fluid. The membrane may be formed from a polymer material that may dissolve upon exposure to blood, water or other fluid compositions. The membrane may retain surgical instruments positioned within the cannula to prevent damage to the interior structures under the skin.

It is also contemplated herein that the sleeve of the cannula may include a material that may harden when inserted into the body or exposed to body heat, saline solution, bodily fluids, oxygen, external heat sources, light sources, etc. The cannula may maintain its hardened shape and may be rigid or semi-rigid. This embodiment may include the use of a removable or dissolvable (i.e. temporary) object of some sort to provide an internal shape for the hardening sleeve. For example, a ballon or a mechanical tamp may be utilized to shape the sleeve, which may then be later removed. In addition, the sleeve may be formed of a shape memory material, such as an alloy or polymer which may become rigid upon exposure to the above elements.

In addition, the cannula contemplated herein may also include, as illustrated in FIG. 11, a proximal stand-off with closeable jaws 1128 that permit partial or complete closure of the sleeve and may allow for minimization of outflow through the cannula. The jaws may be made out of a flexible material or may be formed from a rigid material. As illustrated the teeth of the jaws may be jagged; however, it may also be appreciated that the jaws may not include teeth but may be formed from two smooth mating surfaces.

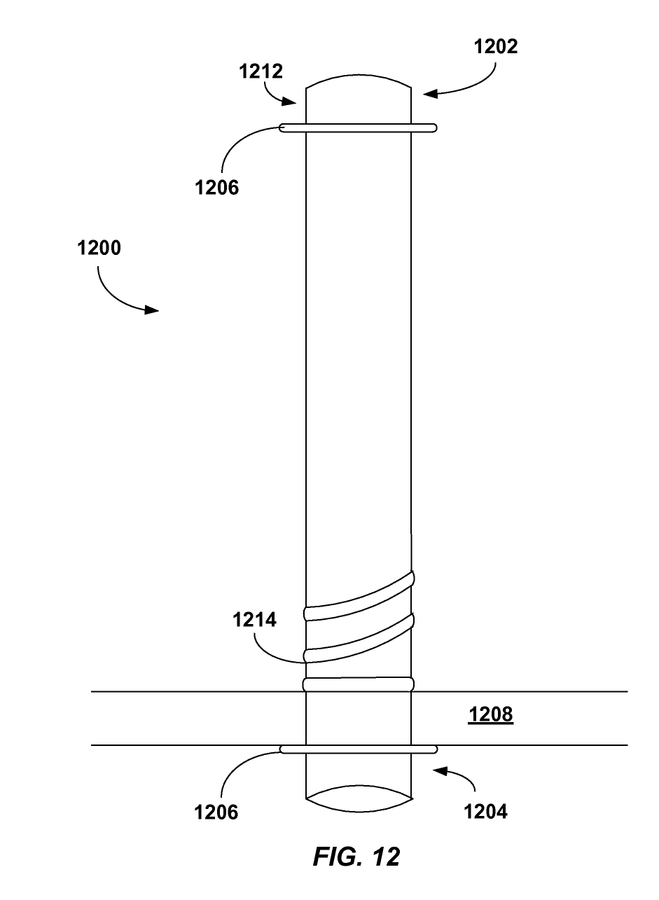

Another embodiment would have an additional shape memory material on the proximal side of the skin wall 1208 so that the cannula may be reversed and thereby transferred intra-articularly from one portal, or incision site, to another. FIG. 12 illustrates an example of such a cannula 1200, which may include a flexible sleeve 1202. The cannula may include shape memory material 1206 near the proximal 1212 and distal ends 1204 of the cannula. One or both ends of the cannula may include a proximal structure 1214, which may maintain tension on the sleeve as described above.

It may be appreciated that in providing such a flexible cannula, the cannula may be directed within the subject. Therefore, a skin incision need not be directly over the deep incision through capsule or muscle. Thus, skin may be cut in advantageous locations for cosmesis, but allow for access to internal structures of interest.

The foregoing description has been presented for purposes of illustration. It may be appreciated that the various features described above may be present individually or combined in a given embodiment or example. It is not intended to be exhaustive or to limit the invention to the precise steps and/or forms disclosed, and obviously many modifications and variations are possible in light of the above teaching. It is intended that the scope of the invention be defined by the claims appended hereto.

What is claimed is:

1. A cannula for providing access into a bodily cavity, comprising:
a flexible sleeve having a proximal portion and a distal portion, wherein said flexible sleeve has a Shore A durometer in the range of 10 to 100; and
a shape memory material affixed to the distal portion of said sleeve.

2. The cannula of claim 1, further comprising a proximal structure affixed to the proximal portion of said sleeve, wherein said proximal structure maintains tension between said distal portion and said proximal portion.

3. The cannula of claim 2, wherein said proximal structure is a spring.

4. The cannula of claim 1, wherein said shape memory material is a shape memory wire.

5. The cannula of claim 1, wherein said shape memory material comprises a metal alloy.

6. The cannula of claim 1, wherein said shape memory material comprises a polymer.

7. The cannula of claim 1, wherein said sleeve comprises a medicament.

8. The cannula of claim 1, further comprising flaps integrated into said distal portion, wherein said flaps remain closed and are configured to open upon the application of force from the interior of the cannula.

9. The cannula of claim 1, further comprising a corrugated portion of said sleeve.

10. The cannula of claim 1, further comprising at least two channels defined within a portion of said sleeve.

11. The cannula of claim 1, further comprising at least one aperture defined in said sleeve.

12. The cannula of claim 1, wherein said sleeve is formed of a mesh material.

13. The cannula of claim 1, wherein said sleeve further comprises a distal end and a membrane affixed to said distal end.

14. The cannula of claim 13, wherein said membrane is configured to dissolved upon exposure to a fluid.

15. The cannula of claim 1, wherein a portion of said sleeve comprises a flexible material configured to harden upon exposure to one or more of the following: body heat, saline solution, bodily fluids, and oxygen.

16. The cannula of claim 15, further comprising a removable or dissolvable object inserted within said portion of said sleeve comprising said flexible material.

17. The cannula of claim 1, further comprising a proximal stand-off and closable jaws.

18. The cannula of claim 1, further comprising a shape memory material affixed to the proximal portion of said sleeve.

19. The cannula of claim 1, wherein said shape memory materials is an o-ring.
